(19) United States
(12) Patent Application Publication
(10) Pub. No.: US 2022/0275407 A1
(43) Pub. Date: Sep. 1, 2022

(54) METHOD FOR PRODUCING JASMONIC ACID BY SOLID STATE FERMENTATION OF MICROORGANISMS

(71) Applicants: WUXI NEW WAY BIOTECH CO.LTD, Wuxi (CN); Jiangnan University, Wuxi (CN)

(72) Inventors: Pu ZHENG, Wuxi (CN); Ziqiang SHEN, Wuxi (CN); Dan WU, Wuxi (CN); Pengcheng CHEN, Wuxi (CN); Ruiying LI, Wuxi (CN)

(73) Assignees: WUXI NEW WAY BIOTECH CO.LTD, Wuxi (CN); Jiangnan University, Wuxi (CN)

(21) Appl. No.: 17/750,037

(22) Filed: May 20, 2022

Related U.S. Application Data
(63) Continuation of application No. PCT/CN2021/108178, filed on Jul. 23, 2021.

(30) Foreign Application Priority Data
Dec. 23, 2020 (CN) 2020115396153

(51) Int. Cl. C12P 7/40

(52) U.S. Cl. CPC C12P 7/40; C12R 2001/645

(57) ABSTRACT

The disclosure discloses a method for producing jasmonic acid by solid state fermentation of microorganisms, and belongs to the field of biotechnology. Lasiodiplodia sp. is inoculated in a solid state fermentation medium containing wheat for static fermentation for 6-15 days, and then, jasmonic acid of 4128 mg/kg wheat can be produced. The method provides a feasibility for extension of production of jasmonic acid by fermentation of microorganisms.

### CROSS-REFERENCE TO RELATED APPLICATIONS

The application claims priority to Chinese patent application No. 202011539615.3, filed on Dec. 23, 2020, the entire contents of which are incorporated herein by reference.

### TECHNICAL FIELD

The disclosure relates to a method for producing jasmonic acid by solid state fermentation of microorganisms, and belongs to the field of biotechnology.

### BACKGROUND

Jasmonic acid (chemical name: 3-oxo-2-2′-cis-pentenyl-cyclopentane-1-acetic acid, molecular formula: C12H18O3) is a new type of plant hormone. Jasmonic acid is contained in vegetative organs such as stem apexes, tender leaves and root tips of higher plants. The jasmonic acid plays a wide range of roles in growth and development of plants, including promotion, induction, inhibition, etc. The jasmonic acid is a signaling molecule in plant defense and stress responses. When plants are physically or chemically damaged, the jasmonic acid induces and activates defense genes in the plants to resist the external damage. In agriculture, the jasmonic acid can be used for improving the drought resistance of plants. In the spice industry, the jasmonic acid and methyl esters thereof can be used as main fragrance components of many fragrant essential oils because of light and elegant fragrance, so as to be applied to production of cigarettes, soaps, chewing gums, etc. In addition, studies have shown that methyl jasmonate has anti-tumor activity and can inhibit the proliferation of various tumor cells.

Preparation methods of the jasmonic acid are mainly extraction from plants, chemical synthesis and microbial fermentation synthesis. However, the content of jasmonic acid in plants is low, and methods for extracting jasmonic acid from plants cannot meet the needs of the current market. The chemical synthesis method has the defects of low reaction yield (30% or less), harsh reaction conditions, racemization of synthesized products, etc., and these defects limit the application range of the jasmonic acid. The method for producing jasmonic acid by microbial fermentation does not have the above defects, so it has attracted people's attention. ALDRIDGE et al. first isolated jasmonic acid from a culture solution of Lasiodiplodia theobroma, indicating that there is a pathway for synthesizing jasmonic acid in some microorganisms. U.S. Patent US20020110881A1 discloses a method for producing jasmonic acid and derivatives thereof by fermentation of Diplodia gossypina. By this technology, the concentration of jasmonic acid obtained by fermentation in a 2.8 L shake flask containing 500 mL of a medium can reach 1.2 g/L. Chinese patent CN 107227264 A discloses a method for producing jasmonic acid by liquid state fermentation of Lasiodiplodia iranensis. By this technology, the maximum concentration of jasmonic acid obtained by fermentation in a 500 mL shake flask containing 100 mL of a medium can reach 1.7 g/L. However, the above methods for producing jasmonic acid by microbial fermentation are all liquid static fermentation. When a stirred fermenter is used for extending a fermentation scale, because the shape of the strain is greatly affected by the shear force, the transfer of oxygen and other nutrients in the fermenter is limited, and mycelial growth is restricted, resulting in a low yield of jasmonic acid.

### SUMMARY

The disclosure discloses a method for producing jasmonic acid by solid state fermentation of microorganisms, including: inoculating a Lasiodiplodia sp. strain in a solid state fermentation medium for static culture for 6-15 days at a culture temperature of 20-35° C. and an air humidity of 80-100%, where a water content of the solid state fermentation medium is 20%-80%, each kg of the solid state fermentation medium contains 200-800 g of a solid substrate, 1-15 g of sodium nitrate, 0.5-3 g of potassium dihydrogen phosphate, 0.1-0.4 g of potassium chloride, 1-9 g of magnesium salt, 0.1-0.9 g of ferric salt, 0.055-5.55 g of calcium chloride and 1-5 g of corn steep liquor, and the solid substrate includes wheat bran, or wheat, or a mixture of wheat bran and wheat.

In an implementation mode of the disclosure, the Lasiodiplodia sp. strain includes Lasiodiplodia iranensis CCTCC NO: M2017288, Lasiodiplodia theobroma MTCC 3068 and mutant strains thereof.

In an implementation mode of the disclosure, the solid state fermentation is performed in a shake flask or a plate or a multi-layer shallow tray fermenter, and an inoculation volume is 8%-15% (v/w). The multi-layer shallow tray fermenter is as follows: 8-12 trays with a diameter of 15-25 cm and a height of 30-50 cm are placed in a 10 L fermenter, these trays are supported by a shallow tray support, a gap between adjacent shallow trays is 0.5-1.5 cm, and each shallow tray is provided with 1-4 overflow ports with a height of 0.15-1.5 cm. A pipeline for ventilation is equipped in the multi-layer shallow tray fermenter, pipeline walls are equipped with air outlets with the same spacing and increasing sizes, and the air introduced into the fermenter is sterile air.

In the disclosure, the jasmonic acid is produced by solid state fermentation of Lasiodiplodia sp. The method of the disclosure is simple in operation. Compared with the form of liquid state fermentation, the method of the disclosure can provide more attachment sites for the growth of strains and has little damage to the strains.

In the disclosure, the Lasiodiplodia sp. is inoculated in a shake flask with 100 g of a solid state fermentation medium for static fermentation at 28° C. for 12 days, and then, jasmonic acid with 4128 mg/kg wheat may be produced.

A fermentation scale of the liquid state fermentation is difficult to extend. In the disclosure, the 10 L multi-layer shallow tray fermenter is used for solid state fermentation at 28° C. for 12 days, and the yield of the jasmonic acid may reach 2698 mg/kg wheat. Compared with the liquid state fermentation, the fermentation cost of each batch of fermentation is reduced by about 40%, and the water consumption is reduced by 0.75 L.

### BRIEF DESCRIPTION OF DRAWINGS

FIG. 1 shows comparison between a mass spectrum (GC-MS) of a product obtained by methyl esterification of the jasmonic acid obtained by methanol extraction in Example 7 and a mass spectrum (GC-MS) of CAS #1211-29-6 methyl jasmonate.

FIG. 2 shows a structural diagram of a 10 L shallow tray fermenter.

### DETAILED DESCRIPTION OF THE EMBODIMENTS

Method for Analyzing Jasmonic Acid Products:

Gas chromatography-mass spectrometer (GC-MS): a product after methyl esterification is identified by a GC-MS. Referring to methods in literatures, operating parameters: the gas chromatographic column is AB-5MS 30 m×0.25 mm×0.25 μm, the carrier gas is helium, the pressure is 130 kg/cm², the sub-gauge pressure is 0.31 kg/cm², and a flow rate is 0.8 mL/min. The temperature of the chromatographic column is increased from an initial temperature of 50° C. to 180° C. at 20° C./min and maintained for 4 min, and then increased to 290° C. at 10° C./min and maintained for 15 min, sample injection is performed at 250° C. without splitting, and the injection volume is 1 μL.

High performance liquid chromatography (HPLC): the concentration of jasmonic acid in a methanol extract is detected with HPLC, referring to methods in literatures. The mobile phase is methanol: 0.1% phosphoric acid=55:45(v/v); the injection volume is 20 μL; the flow rate is 1 mL/min; and the detector is an ultraviolet detector.

Potato dextrose agar (PDA) medium includes 200 g of potatoes, 20 g of dextrose, 15-20 g of agar, and 1000 mL of distilled water.

Potato dextrose broth (PDB) medium includes 200 g of potatoes, 20 g of dextrose and 1000 mL of distilled water.

Specific Implementation Mode

### Example 1: Solid State Fermentation of Jasmonic Acid Using Solid State Fermentation Mediums Containing Different Solid Substrates

Lasiodiplodia iranensis CCTCC NO: M2017288 strain is activated by PDA medium, and then inoculated in a 250 mL shake flask with 100 g of a sterilized solid state fermentation medium in an inoculation volume of 10% (v/w) for static fermentation culture in an incubator at 28° C. for 12 days. A formula of the solid state fermentation medium includes: 20 g of a solid substrate (the solid substrate refers to bagasse or corn straw or soybean meal or wheat bran or wheat), 3.75 g of sucrose, 0.3375 g of sodium nitrate, 0.15 g of potassium dihydrogen phosphate, 0.03 g of potassium chloride, 0.45 g of magnesium sulfate heptahydrate, 0.0675 g of ferrous sulfate heptahydrate, 0.1125 g of corn steep liquor, and 0.75 mL of a trace element solution. Water is added to enable the initial water content of the solid state fermentation medium to reach 80% (the initial water content of the solid state fermentation medium refers to a percentage of the mass of liquid substances other than the solid substrate in the solid state fermentation medium to the mass of the solid state fermentation medium). The trace element solution contains: 0.03 g/L zinc sulfate heptahydrate, 0.003 g/L manganese sulfate heptahydrate, 0.003 g/L copper sulfate heptahydrate, and 0.003 g/L sodium molybdate dihydrate. After the end, the jasmonic acid is extracted from the fermented mash with methanol, and the concentration of the jasmonic acid is determined by HPLC. The results are shown in Table 1.

TABLE 1
The influence of different type of solid substrate in
solid state fermentation medium on yield of jasmonic
acid produced by solid state fermentation of strain

| | Yield of jasmonic acid |
|---|---|
| Type of solid substrate | (mg/kg solid substrate) |
| Bagasse | 0 |
| Soybean meal | 13 |
| Corn straw | 6 |
| Wheat | 945 |
| Wheat bran | 658 |

When the solid substrate is wheat bran or wheat, the yield of the jasmonic acid is higher and can reach 600 mg/kg solid substrate or more.

### Example 2: Solid State Fermentation of Jasmonic Acid Using Solid State Fermentation Mediums with Different Initial Water Contents

Lasiodiplodia iranensis CCTCC NO: M2017288 strain is activated by PDA medium, and then inoculated in a 250 mL shake flask with 100 g of a sterilized solid state fermentation medium in an inoculation volume of 10% (v/w) for static fermentation culture in an incubator at 28° C. for 12 days. A formula of the solid state fermentation medium used includes: wheat, 3.75 g of sucrose, 0.3375 g of sodium nitrate, 0.15 g of potassium dihydrogen phosphate, 0.03 g of potassium chloride, 0.45 g of magnesium sulfate heptahydrate, 0.0675 g of ferrous sulfate heptahydrate, 0.1125 g of corn steep liquor, and 0.75 mL of a trace element solution. The trace element solution contains: 0.03 g/L zinc sulfate heptahydrate, 0.003 g/L manganese sulfate heptahydrate, 0.003 g/L copper sulfate heptahydrate, and 0.003 g/L sodium molybdate dihydrate. Meanwhile, the initial water content of the solid state fermentation medium is set to 40%, 50%, 60%, 65%, 70%, 75%, or 80% respectively (when the initial water content is 40%, 100 g of the solid state fermentation medium contains 60 g of wheat; and when the initial water content is 50%, 100 g of the solid state fermentation medium contains 50 g of wheat). After the culture, the jasmonic acid is extracted from the fermented mash with methanol, and the concentration of the jasmonic acid is determined by HPLC. The results are shown in Table 2.

TABLE 2
The influence of initial water content of solid state
fermentation medium on yield of jasmonic acid produced
by solid state fermentation of strain

| Initial water content of solid | Yield of jasmonic acid |
|---|---|
| state fermentation medium | (mg/kg wheat) |
| 40% | 78 |
| 50% | 65 |
| 60% | 122 |
| 65% | 785 |
| 70% | 1622 |
| 75% | 1701 |
| 80% | 945 |

It can be seen from Table 2 that the initial water content of the solid state fermentation medium has a significant influence on the yield of jasmonic acid produced by fermentation of a strain. When the initial water content is 60% or less, the yield of the jasmonic acid is low. If the initial water content is too high, the yield of the jasmonic acid will be reduced. For example, when the water content is 80% or more, the yield of the jasmonic acid decreases. In this example, when the initial water content of the solid state fermentation medium is set to 75%, the yield of the jasmonic acid reaches the maximum value.

### Example 3 Solid State Fermentation of Jasmonic Acid Using Solid State Fermentation Mediums Containing Different Concentrations of CaCl₂

Lasiodiplodia iranensis CCTCC NO: M2017288 strain is activated by PDA medium, and then inoculated in a 250 mL shake flask with 100 g of a sterilized solid state fermentation medium in an inoculation volume of 10% (v/w) for static fermentation culture in an incubator at 28° C. for 12 days. A formula of the solid state fermentation medium used includes: 25 g of wheat, 3.75 g of sucrose, 0.3375 g of sodium nitrate, 0.15 g of potassium dihydrogen phosphate, 0.03 g of potassium chloride, 0.45 g of magnesium sulfate heptahydrate, 0.0675 g of ferrous sulfate heptahydrate, 0 g, 0.004 g, 0.021 g, 0.042 g, 0.062 g or 0.416 g of calcium chloride, 0.1125 g of corn steep liquor, 0.75 mL of a trace element solution, and 75% initial water content. The trace element solution contains: 0.03 g/L zinc sulfate heptahydrate, 0.003 g/L manganese sulfate heptahydrate, 0.003 g/L copper sulfate heptahydrate, and 0.003 g/L sodium molybdate dihydrate. After the culture, the jasmonic acid is extracted from the fermented mash with methanol, and the concentration of the jasmonic acid is determined by HPLC. The results are shown in Table 3.

TABLE 3
The influence of CaCl₂ on yield of jasmonic acid
produced by solid state fermentation of strain

| CaCl₂ concentration | Yield of jasmonic acid |
|---|---|
| (g/kg_{solid state fermentation medium}) | (mg/kg wheat) |
| 0 | 1701 |
| 0.04 | 2066 |
| 0.21 | 2489 |
| 0.42 | 2287 |
| 0.62 | 2253 |
| 4.16 | 1920 |

It can be seen from Table 3 that CaCl₂ in the solid state fermentation medium has a promoting effect on the production of jasmonic acid by fermentation of the strain, and with the gradual increase of the concentration of CaCl₂, the promoting effect first increases and then decreases. When the concentration of CaCl₂ is set to 0.21 g/kg_{solid state fermentation medium}, the promoting effect is the most significant, and the yield of the jasmonic acid reaches 2489 mg/kg wheat, which is 46.32% higher than that without CaCl₂.

### Example 4 Solid State Fermentation of Jasmonic Acid Using Solid State Fermentation Mediums Containing Different Inorganic Salts

Lasiodiplodia iranensis CCTCC NO: M2017288 strain is activated by PDA medium, and then inoculated in a 250 mL shake flask with 100 g of a sterilized solid state fermentation medium in an inoculation volume of 10% (v/w) for static fermentation culture in an incubator at 28° C. for 12 days. A formula of the solid state fermentation medium used includes: 75% initial water content, 25 g of wheat, 3.75 g of sucrose, 0.3375 g of sodium nitrate, 0.021 g of CaCl₂), 0.1125 g of corn steep liquor, and inorganic salts. The inorganic salts adopt any one of a control group, an experimental group 1, an experimental group 2, an experimental group 3, an experimental group 4, and an experimental group 5 as follows.

The inorganic salts contained in the solid state fermentation medium in the control group are (per kg of solid state fermentation medium): 1.5 g of potassium dihydrogen phosphate, 0.3 g of potassium chloride, 4.5 g of magnesium sulfate heptahydrate, 0.675 g of ferrous sulfate heptahydrate, and 7.5 mL of trace elements.

The inorganic salts contained in the solid state fermentation medium in the experimental group 1 are (per kg of solid state fermentation medium): 1.5 g of potassium dihydrogen phosphate, 0.3 g of potassium chloride, 4.5 g of magnesium sulfate heptahydrate, and 0.675 g of ferrous sulfate heptahydrate.

The inorganic salts contained in the solid state fermentation medium in the experimental group 2 are (per kg of solid state fermentation medium): 0.3 g of potassium chloride, 4.5 g of magnesium sulfate heptahydrate, and 0.675 g of ferrous sulfate heptahydrate.

The inorganic salts contained in the solid state fermentation medium in the experimental group 3 are (per kg of solid state fermentation medium): 1.5 g of potassium dihydrogen phosphate, 4.5 g of magnesium sulfate heptahydrate, and 0.675 g of ferrous sulfate heptahydrate.

The inorganic salts contained in the solid state fermentation medium in the experimental group 4 are (per kg of solid state fermentation medium): 1.5 g of potassium dihydrogen phosphate, 0.3 g of potassium chloride, and 0.675 g of ferrous sulfate heptahydrate.

The inorganic salts contained in the solid state fermentation medium in the experimental group 5 are (per kg of solid state fermentation medium): 1.5 g of potassium dihydrogen phosphate, 0.3 g of potassium chloride, and 4.5 g of magnesium sulfate heptahydrate.

After the culture, the jasmonic acid is extracted from the fermented mash with methanol, and the concentration of the jasmonic acid is determined by HPLC. The results are shown in Table 4.

TABLE 4
The influence of inorganic salts on yield of jasmonic
acid produced by solid state fermentation of strain

| Experimental group | Yield of jasmonic acid (mg/kg wheat) |
|---|---|
| Control group | 2489 |
| Experimental group 1 | 3684 |
| Experimental group 2 | 3668 |
| Experimental group 3 | 3148 |
| Experimental group 4 | 745 |
| Experimental group 5 | 1631 |

It can be seen from Table 4 that the solid state fermentation medium is supplemented with different nutrient combinations based on the medium including 25 g of wheat, 75% initial water content and 0.21 g/kg CaCl₂. By comparing the control group with the experimental group 1, it can be seen that the trace elements inhibit the production of jasmonic acid by the strain. By comparing the experimental group 1 with the experimental group 3, it can be seen that the appropriate addition of potassium chloride is conducive to the production of jasmonic acid by the strain. By comparing the experimental group 1 with the experimental group 2, it can be seen that the presence or absence of potassium dihydrogen phosphate has a less influence on the yield of jasmonic acid. The yields of the experimental group 4 and the experimental group 5 are significantly lower than the yield of the experimental group 1, indicating that the magnesium sulfate heptahydrate and the ferrous sulfate heptahydrate are crucial for the production of jasmonic acid by the strain. Therefore, it is recommended that the solid state fermentation medium contains 1.5 g/kg potassium dihydrogen phosphate, 0.3 g/kg potassium chloride, 4.5 g/kg magnesium sulfate heptahydrate, and 0.675 g/kg ferrous sulfate heptahydrate.

### Example 5 Solid State Fermentation of Jasmonic Acid Using Solid State Fermentation Mediums Containing Different Additional Carbon Sources

Lasiodiplodia iranensis CCTCC NO: M2017288 strain is activated by PDA medium, and then inoculated in a 250 mL shake flask with 100 g of a sterilized solid state fermentation medium in an inoculation volume of 10% (v/w) for static fermentation culture in an incubator at 28° C. for 12 days. A formula of the solid state fermentation medium used includes: 75% initial water content, 25 g of wheat, 0.3375 g of sodium nitrate, 0.15 g of potassium dihydrogen phosphate, 0.03 g of potassium chloride, 0.45 g of magnesium sulfate heptahydrate, 0.0675 g of ferrous sulfate heptahydrate, 0.021 g of calcium chloride, and 0.1125 g of corn steep liquor. At the same time, additional carbon sources in the solid state fermentation medium are adjusted with reference to Table 5 (as shown in Table 5: no additional carbon source or 3.75 g of sucrose, 37.5 g of dextrose, 37.5 g of fructose, 2.92 g of linolenic acid or 2.92 g of linoleic acid). After the culture, the jasmonic acid is extracted from the fermented mash with methanol, and the concentration of the jasmonic acid is determined by HPLC. The results are shown in Table 5.

TABLE 5
The influence of different additional carbon sources on yield
of jasmonic acid produced by solid state fermentation of strain

| Carbon source | Yield of jasmonic acid |
|---|---|
| (g/kg_{solid state fermentation medium}) | (mg/kg wheat) |
| Control | 4182 |
| (no carbon source) | |
| 37.5 g of sucrose | 3684 |
| 37.5 g of dextrose | 2213 |
| 37.5 g of fructose | 3584 |
| 2.92 g of linolenic acid | 3854 |
| 2.92 g of linoleic acid | 3781 |

It can be seen from Table 5 that the yield of the jasmonic acid can reach a maximum of 4182 mg/kg without additional carbon sources in the solid state fermentation medium, which is 41% higher than the yield of the jasmonic acid obtained by the medium with 37.5 g of dextrose added. Obviously, the yield of the jasmonic acid is more facilitated without additional carbon sources in the solid state fermentation medium.

### Example 6 Solid State Fermentation of Jasmonic Acid Using Petri Dish with Different Loading Weights of Solid State Fermentation Medium

Lasiodiplodia iranensis CCTCC NO: M2017288 strain is activated by PDA medium, and then inoculated in petri dish (150 mm in diameter and 30 mm in height) with different loading weights of solid state fermentation medium in an inoculation volume of 10% (v/w) for static fermentation culture in an incubator at 28° C. for 12 days. A formula of the solid state fermentation medium used includes: 75% initial water content, 25 g of wheat, 3.75 g of sucrose, 0.3375 g of sodium nitrate, 0.15 g of potassium dihydrogen phosphate, 0.03 g of potassium chloride, 0.45 g of magnesium sulfate heptahydrate, 0.0675 g of ferrous sulfate heptahydrate, 0.021 g of calcium chloride, 0.1125 g of corn steep liquor, and 0.75 mL of a trace element solution. The trace element solution contains: 0.03 g/L zinc sulfate heptahydrate, 0.003 g/L manganese sulfate heptahydrate, 0.003 g/L copper sulfate heptahydrate, and 0.003 g/L sodium molybdate dihydrate. After the culture, the jasmonic acid is extracted from the fermented mash with methanol, and the concentration of the jasmonic acid is determined by HPLC. The results are shown in Table 6.

TABLE 6
The influence of different loading weights of medium on yield
of jasmonic acid produced by solid state fermentation of strain

| | Yield of jasmonic acid |
|---|---|
| Loading weight of medium | (mg/kg wheat) |
| Control (100 g of medium/ | 2489 |
| 250 mL shake flask) | |
| 150 g of medium/petri dish | 223 |
| 200 g of medium/petri dish | 3885 |
| 250 g of medium/petri dish | 1725 |

It can be seen from Table 6 that when solid state fermentation of the strain is performed in the petri dish, the loading weight of the medium will significantly affect the final yield of jasmonic acid. In this example, under the condition of 200 g loading weight, the maximum yield (3885 mg/kg wheat) of jasmonic acid is obtained, which is 36% higher than the yield in the shake flask in the control group.

### Example 7 Production of Jasmonic Acid by Fermentation with 10 L Shallow Tray Fermenter

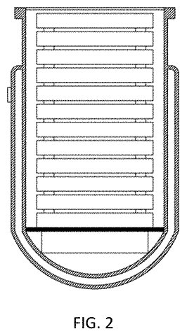

As shown in FIG. 2, a 10 L shallow tray fermenter is a multi-layer shallow tray fermenter as follows: 8-12 trays with a diameter of 15-25 cm and a height of 30-50 cm are placed in a 10 L fermenter, these trays are supported by a shallow tray support, a gap between adjacent shallow trays is 0.5-1.5 cm, and each shallow tray is equipped with 1-4 overflow ports with a height of 0.15-1.5 cm. A pipeline for ventilation is equipped in the multi-layer shallow tray fermenter, pipeline walls are provided with air outlets with the same spacing and increasing sizes, and the air introduced into the fermenter is sterile air.

A formula of the solid state fermentation medium used includes: 75% initial water content, 25 g of wheat, 0.3375 g of sodium nitrate, 0.15 g of potassium dihydrogen phosphate, 0.03 g of potassium chloride, 0.45 g of magnesium sulfate heptahydrate, 0.0675 g of ferrous sulfate heptahydrate, 0.021 g of calcium chloride, and 0.1125 g of corn steep liquor. The solid substrate in the solid state fermentation medium is placed in the shallow tray fermenter, the solid substrate and the fermenter are sterilized at the same time, and the other components in the solid state fermentation medium are sterilized separately.

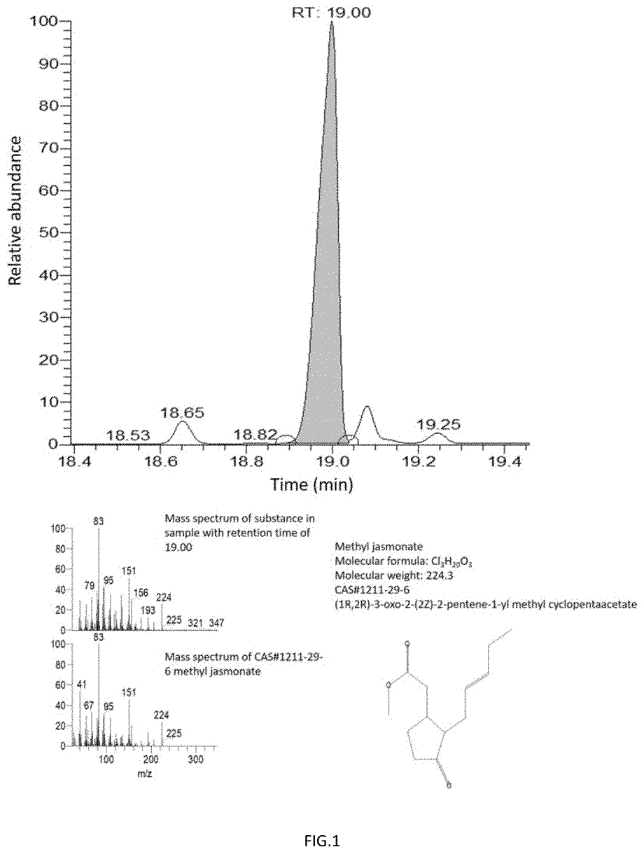

Lasiodiplodia iranensis CCTCC NO: M2017288 strain is activated by PDB medium, and then, a seed solution obtained by activation is inoculated in the other components in the sterilized solid state fermentation medium in an inoculation volume of 8% (v/w). The other components in the solid state fermentation medium and the seed solution are supplemented into each shallow tray through a feeding port of the fermenter and the overflow ports on the shallow tray for fermentation. In the fermentation process, sterile air is introduced into the fermenter, an air humidity is maintained at 95%, and a temperature is maintained at 28° C. After static culture for 12 days, the jasmonic acid is extracted from the fermented mash with methanol, and the concentration of the jasmonic acid is determined by HPLC. The result shows that the yield of the jasmonic acid is 2698 mg/kg wheat. The jasmonic acid is identified by a GC-MS (FIG. 1), and the result shows that the structure of the product is 3-oxo-2-2′-cis-pentenyl-cyclopentane-1-acetic acid.

### Comparative Example 1 Production of Jasmonic Acid by Liquid State Submerged Fermentation

Liquid state submerged fermentation: a Lasiodiplodia iranensis CCTCC NO: M2017288 strain is activated by PDB medium to obtain a seed solution, and the seed solution is inoculated in a sterilized liquid fermentation medium according to 8% (v/v) (the liquid fermentation medium includes: 25-500 g/L sucrose, 1-15 g/L sodium nitrate, 0.5-3 g/L potassium dihydrogen phosphate, 0.1-0.4 g/L potassium chloride, 1-9 g/L magnesium sulfate heptahydrate, 0.1-0.9 g/L ferric sulfate heptahydrate, 1-5 g/L corn steep liquor, 0.2-20 g/L soybean oil, and 10-12 ml/L trace elements, and a pH is 4.5-8; and the trace elements include: 0.01-0.04 g/L ferric sulfate heptahydrate, 0.001-0.004 g/L manganese sulfate heptahydrate, 0.001-0.004 g/L copper sulfate heptahydrate, and 0.001-0.004 g/L sodium molybdate dihydrate.). In the fermentation process, an aeration rate is set to 0.1 vvm, and a temperature is controlled at 28° C. Static culture is performed for 12 days, and after the fermentation, the concentration of the product is determined by HPLC.

The results show that each batch can produce 0.98 g of jasmonic acid through liquid state submerged fermentation of the Lasiodiplodia iranensis CCTCC NO: M2017288 in the fermenter. However, Example 7 adopts solid state shallow tray fermentation, and each batch can obtain 2.02 g of jasmonic acid. The yield of solid state shallow tray fermentation is 51% higher than that of liquid state submerged fermentation.

### Comparative Example 2 Production of Jasmonic Acid by Liquid State Shallow Tray Fermentation

The specific implementation mode of solid state shallow tray fermentation is the same as that in Example 7.

Liquid state shallow tray fermentation: The 10 L shallow tray fermenter which is the same as that in Example 7 is used. The fermenter is sterilized separately, and the liquid fermentation medium (the formula of the liquid fermentation medium is the same as that in Comparative Example 1) is also sterilized separately. Lasiodiplodia iranensis CCTCC NO: M2017288 strain is activated by PDB medium to obtain a seed solution, and the seed solution is inoculated in a sterilized liquid fermentation medium according to 8% (v/v). The liquid fermentation medium and the seed solution are supplemented into each shallow tray through a feeding port and the overflow ports of the fermenter for fermentation. In the fermentation process, an aeration rate is set to 0.1 vvm, and a temperature is controlled at 28° C. Static culture is performed for 7 days, and after the fermentation, the concentration of the product is determined by HPLC. The results show that by using the shallow tray fermenter, each batch in liquid state fermentation and solid state fermentation can respectively obtain 2.10 g and 2.02 g of jasmonic acid.

However, the liquid state shallow tray fermentation requires sucrose, the price is 40.76 yuan/kg, and 150 g of sucrose is used for each batch, that is, 6.114 yuan. The solid state shallow tray fermentation does not require the sucrose but requires wheat, the price is 4 yuan/kg, and 750 g of wheat are used for each batch, that is, 3 yuan which is significantly lower than that in the liquid state shallow tray fermentation. The liquid state shallow tray fermentation requires trace elements, and the cost of each batch of trace elements is 0.344 yuan. The solid state shallow tray fermentation does not require the trace elements. Additional organic nitrogen sources, inorganic nitrogen sources and inorganic salts used in the solid state shallow tray fermentation are 75% of the amounts required for the liquid state shallow tray fermentation, so that each batch of fermentation can save 1.037 yuan. The water consumption per batch of solid state shallow tray fermentation is 2.25 L, and the water consumption per batch of liquid state shallow tray fermentation is 3.00 L. In conclusion, compared with the liquid state fermentation, the cost of each batch of solid state fermentation of jasmonic acid is reduced by about 40%, and the water consumption is reduced by 0.75 L. Therefore, the solid state fermentation has the advantages of low raw material price, low water consumption and the like, and is beneficial to realize the industrialized production of jasmonic acid.

What is claimed is:

1. A method for producing jasmonic acid by solid state fermentation of microorganisms, comprising: inoculating Lasiodiplodia sp. in a solid state fermentation medium for static culture for 6-15 days at a culture temperature of 20-35° C. and an air humidity of 80-100%, where in a water content of the solid state fermentation medium is 20-80%, each kg of the solid state fermentation medium contains 200-800 g of a solid substrate, 1-15 g of sodium nitrate, 0.5-3 g of potassium dihydrogen phosphate, 0.1-0.4 g of potassium chloride, 1-9 g of magnesium salt, 0.1-0.9 g of ferric salt, 0.055-5.55 g of calcium chloride and 1-5 g of corn steep liquor, and the solid substrate is wheat bran, wheat, or a mixture of wheat bran and wheat.

2. The method for producing jasmonic acid by solid state fermentation according to claim 1, wherein the Lasiodiplodia sp. comprises Lasiodiplodia iranensis CCTCC NO: M2017288, Lasiodiplodia theobroma MTCC 3068 and mutant strains thereof.

3. The method for producing jasmonic acid by solid state fermentation according to claim 2, wherein a formula of the solid state fermentation medium used comprises: 75% initial water content, 250 g/kg wheat, 3.375 g/kg sodium nitrate, 1.5 g/kg potassium dihydrogen phosphate, 0.3 g/kg potassium chloride, 4.5 g/kg magnesium sulfate heptahydrate, 0.675 g/kg ferrous sulfate heptahydrate, 0.21 g/kg calcium chloride and 1.125 g/kg corn steep liquor.

4. The method for producing jasmonic acid by solid state fermentation according to claim 1, wherein surface fermentation is performed in a shake flask or a petri dish or a multi-layer shallow tray fermenter.

5. The method for producing jasmonic acid by solid state fermentation according to claim 1, wherein a fermentation inoculation volume is 8%-15% (v/w).

6. The method for producing jasmonic acid by solid state fermentation according to claim 4, wherein the multi-layer shallow tray fermenter is as follows: 8-12 trays with a diameter of 15-25 cm and a height of 30-50 cm are placed in a 10 L fermenter, these trays are supported by a shallow tray support, a gap between adjacent shallow trays is 0.5-1.5 cm, and each shallow tray is equipped with 1-4 overflow ports with a height of 0.15-1.5 cm.

7. The method for producing jasmonic acid by solid state fermentation according to claim 6, wherein a pipeline for ventilation is equipped in the multi-layer shallow tray fermenter, pipeline walls are equipped with air outlets with the same spacing and increasing sizes, and the air introduced into the fermentater is sterile air.
